(19) United States
(12) Patent Application Publication
(10) Pub. No.: US 2008/0015377 A1
(43) Pub. Date: Jan. 17, 2008

(54) METHODS OF MAKING AN ANTISTATIC AGENT

(75) Inventors: Theodorus Lambertus Hoeks, Bergen Op Zoom (NL); Chiel Albertus Leenders, Fijnaart (NL); Robert Dirk van de Grampel, Tholen (NL)

Correspondence Address: CANTOR COLBURN LLP - GE PLASTICS - SMITH, 55 GRIFFIN RD SOUTH, BLOOMFIELD, CT, 06002, US

(73) Assignee: GENERAL ELECTRIC COMPANY, Schenectady, NY (US)

(21) Appl. No.: 11/772,309

(22) Filed: Jul. 2, 2007

Related U.S. Application Data
(62) Division of application No. 10/983,878, filed on Nov. 8, 2004.

(51) Int. Cl. C07C 309/64

(52) U.S. Cl. 558/51

(57) ABSTRACT

Disclosed are methods for making a phosphonium sulfonate salt of generic formula (1): 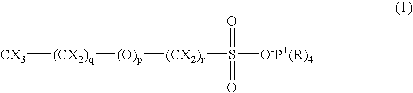 wherein each X is independently a halogen or hydrogen, provided that the molar ratio of halogen to hydrogen is greater than- about 0.90; p is 0 or 1 and q and r are integers of 0 to about 7 provided that q+r is less than 8 and that if p is 1 then r is greater than zero; and each R is the same or different hydrocarbon radical containing 1 to about 18 carbon atoms.

### CROSS REFERENCE TO RELATED APPLICATIONS

This application is a divisional application of U.S. patent application Ser. No. 10/983,878 filed on Nov. 8, 2004, which is incorporated herein in its entirety.

### BACKGROUND OF THE INVENTION

This disclosure relates to a method of making an antistatic agent.

Thermoplastics are useful in the manufacture of articles and components for a wide range of applications, from automotive parts to electronic appliances. Because of their broad use, particularly in electronic applications, it is desirable to provide thermoplastic resins with antistatic agents. Many polymers or blends of polymers are relatively non-conductive, which can lead to static charge build-up during processing and use of the polymer. Charged molded parts, for example, may attract small dust particles, and may thus interfere with a smooth surface appearance, for example by causing a decrease in the transparency of the article. In addition, the electrostatic charge may be a serious obstacle in the production process of such polymers.

Anti-static agents are materials that are added to polymers to reduce their tendency to acquire an electrostatic charge, or, when a charge is present, to promote the dissipation of such a charge. Organic anti-static agents are usually hydrophilic or ionic in nature. When present on the surface of polymeric materials, they facilitate the transfer of electrons and thus eliminate the build up of a static charge. Anti-static agents have also been added to the polymer composition before further processing into articles, and may thus be referred to as “internally applied.” Useful anti-static agents applied in this maimer are thermally stable and able to migrate to the surface during processing.

A large number of anti-static agents having surfactants as their main constituent have been considered and tried. Many suffer from one or more drawbacks, such as lack of compatibility with the polymer (which interferes with uniform dispersibility), poor heat stability, and/or poor antistatic characteristics. Poor heat resistance in particular can adversely affect the optical properties of engineering thermoplastic such as aromatic polycarbonates.

Particular phosphonium salts of certain sulfonic acids, however have been shown to be useful antistatic agents. U.S. Pat. No. 4,943,380 discloses reducing the static charge on polycarbonate resins with an anti-static composition containing 90-99.9 weight % of polycarbonate and 0.1-10 weight % of a heat resistant phosphonium sulfonate having the general formula: 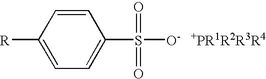
wherein R is a straight or branched chain alkyl group having 1 to 18 carbon atoms; R₁, R₂ and R₃ are the same, each being an aliphatic hydrocarbon having 1 to 8 carbon atoms or an aromatic hydrocarbon group having 6 to 12 carbon atoms; and R₄ is a hydrocarbon group having 1 to 18 carbon atoms.

U.S. Pat. No. 6,194,497 discloses antistatic resin compositions, particularly transparent resin compositions, comprising a thermoplastic polymer and a halogenated medium- or short-chain alkylsulfonic acid salt of a tetrasubstituted phosphonium cation. The antistatic agent described therein is prepared by ion exchange of a potassium haloalkylsulfonate to produce the corresponding acid. The haloalkylsulfonic acid is then reacted with tetrabutylphosphonium hydroxide to product the antistatic agent.

An advantage of this synthesis is that use of an ion exchange step during synthesis results in a product that is very pure, i.e., contains little to no halogenated compounds that may ultimately lead to degradation of resins such as polycarbonates. However, while suitable for its intended purposes, this particular synthesis also has a number of drawbacks. For example, use of an ion exchange step increases the expense of the process, and may lead to the production of waste requiring disposal procedures. The synthesis also uses the potassium salt as a starting product, which is prepared from the corresponding sulfonylfluoride. Since the solubility of potassium peralkylsulfonates is relatively low. e.g., on the order of 5% at 20° C. a water/ethanol mixture is needed in the ion exchange. The flammability of ethanol requires the implementation of significant safety precautions during the synthesis. In addition, selecting the appropriate water/ethanol ratio is also important. An excess of alcohol may render the final product soluble in the reaction solvent, such that isolation of the product may require a further extraction step.

There accordingly remains a demand in the art for more efficient processes, particularly one-step processes, for making phosphonium sulfonate antistatic agents, as well as thermoplastic resin compositions that incorporate these antistatic agents. It would further be desirable for such processes to produce the antistatic agent in good yields without having a detrimental effect on the safety of the process and/or the purity of the product.

### BRIEF SUMMARY OF THE INVENTION

The above-described and other deficiencies of the art are met by a method of making a phosphonium sulfonate salt of formula (1): 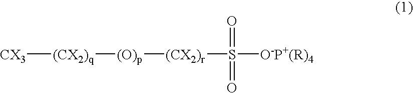
wherein each X is independently a halogen or hydrogen, provided that the molar ratio of halogen to hydrogen is greater than about 0.90; p is 0 or 1 and q and r are integers of 0 to about 7, provided that q+r is less than 8 and that if p is not zero then r is greater than zero; and each R is independently a hydrocarbon radical having 1 to about 18 carbon atoms, the method comprising combining in an aqueous medium a compound of the formula (2): 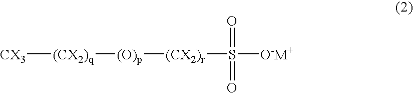
wherein M is K, and X, q, p, and r are as defined above, with a compound of the formula (3):
(R)₄P-Z (3)
wherein Z is a halogen and R is as defined above; and separating the product of formula (1) from the aqueous medium.

In another embodiment, a method of making the phosphonium sulfonate salt of formula (1) comprises first combining in an aqueous medium, a compound of the formula (4) 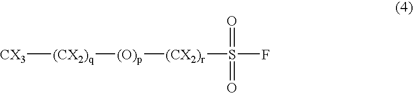
with potassium hydroxide, and a stoichiometric excess of a compound of the generic formula (3):
(R)₄P-Z (3)
wherein X, p, q, r, and R have the same meanings as in formula (1), and Z is a halogen; and separating the product of formula (1) from the aqueous medium.

Another embodiment comprises an antistatic agent of formula (1) made by one of the foregoing methods.

In another embodiment there are provided thermoplastic compositions comprising a thermoplastic polymer and an antistatic agent made by one of the foregoing methods.

### DETAILED DESCRIPTION OF THE INVENTION

It has been unexpectedly found by the inventors hereof that a phosphonium haloalkylsulfonate salt suitable for use as antistatic agent may be readily obtained in aqueous medium in one step from the corresponding tetraalkylphosphonium halide and potassium haloalkylsulfonate salt. The phosphonium haloalkylsulfonate salt may be formed in a process conducted at about 15° C. to about 100° C. Alternatively, the phosphonium haloalkylsulfonate salt may be obtained in aqueous medium in one step from the corresponding tetraalkylphosphonium halide, the haloalkylsulfonyl fluoride, and potassium hydroxide, wherein the potassium haloalkylsulfonate may be prepared in situ. The reactants are readily available, and use of water as the reaction solvent expedites isolation of the product. Thus, in a surprising and highly advantageous feature, the inventors hereof have found that a simple mixing of the reactants may result in a precipitation of the targeted anti-static molecule in high yields.

In general, the phosphonium haloalkylsulfonate salts are of the generic formula (1): 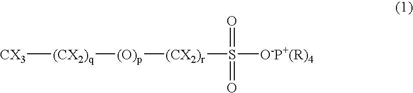
wherein X is independently selected from halogen or hydrogen, provided that the molar ratio of halogen to hydrogen is greater than about 0.90. The halogens may be independently selected from bromine, chlorine, fluorine, and iodine. Specifically, the halogen is fluorine.

Further in formula (1), p is zero or one, and q and r are integers of 0 to about 7, provided that q+r is less than 8 and that if p is not zero then r is greater than zero. In one embodiment, p is zero.

Each R in formula (1) is independently a hydrocarbon radical containing 1 to about 18 carbon atoms, that is, each R is the same or different, and may be a straight or branched chain aliphatic hydrocarbon radical containing 1 to about 18 carbon atoms, or an aromatic hydrocarbon radical containing 6 to about 18 carbon atoms. As used herein, an “aromatic” radical is inclusive of fully aromatic radicals, aralkyl radicals, and alkaryl radicals. In one embodiment, three of the R groups in the organic phosphonium cation may be the same aliphatic hydrocarbon radical containing 1 to about 8 carbon atoms or aromatic hydrocarbon radical containing 6 to about 12 carbon atoms, while the fourth R group may be a hydrocarbon radical containing 1 to about 18 carbon atoms.

The antistatic agent may thus be a highly halogenated phosphonium sulfonate salt containing an organic sulfonate anion and a tetrasubstituted organic phosphonium cation. Specific examples are perfluorinated salts. It is to be understood that perfluorinated salts, due to the fluorination method (electrolysis), may include only partially fluorinated components.

Specific examples of suitable organic sulfonate anions include perfluoromethane sulfonate, perfluoroethane sulfonate, perfluoropropane sulfonate, perfluorobutane sulfonate, perfluoropentane sulfonate, perfluorohexane sulfonate, perfluoroheptane sulfonate, and perfluorooctane sulfonate. Combinations of the foregoing may also be used.

Examples of specific phosphonium cations include cations such as tetramethyl phosphonium, tetraethyl phosphonium, tetra-n-propyl phosphonium, tetraisopropyl phosphonium, tetrabutyl phosphonium, triethylmethyl phosphonium, tributylmethyl phosphonium, tributylethyl phosphonium, trioctylmethyl phosphonium, trimethylbutyl phosphonium, trimethyloctyl phosphonium, trimethyllauryl phosphonium, trimethylstearyl phosphonium, triethyloctyl phosphonium, tetraphenyl phosphonium, triphenylmethyl phosphonium, triphenylbenzyl phosphonium, and tributylbenzyl phosphonium. Combinations of the foregoing may also be used.

In one embodiment there is provided a method for making the phosphonium sulfonates of formula (1) comprising combining, in an aqueous medium, at elevated temperatures of about 50° C. to about 100° C., a compound of the formula (2): 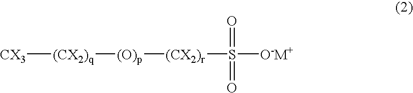
wherein M is potassium, and X, q, p, and r are as defined above, with a stoichiometric excess of a compound of the formula (3):
(R)₄P-Z (3)
wherein Z is a halogen and R is as defined above; and separating the product of formula (1). Specifically Z may be bromine or chlorine.

In one manner of proceeding, the process may comprise a perhaloalkylsulfonate potassium salt of formula (2) in an aqueous medium. It has been surprisingly found that the potassium salt of (2) is fully soluble in water at about 85° C., obviating the need for a cosolvent. The aqueous medium, therefore, may be substantially free of a cosolvent such as ethanol, for example. As used herein, “an aqueous medium” means a solution, dispersion, or suspension of the perhaloalkylsulfonate salt in water. Further as used herein, an aqueous medium “substantially free of a cosolvent” means an aqueous medium containing less than about 1, specifically less than about 0.5, and more specifically less than about 0.1 volume percent cosolvent. While the use of a cosolvent is possible, the use of water substantially free of a cosolvent results in a higher purity product, and avoids the safety concerns that arise from use of volatile solvents. Suitable cosolvents, when used, may aid in dissolving the sulfonate alkali salts, and include lower alcohols such as methanol, ethanol, and the like, and chlorinated solvents such as dichloromethane, and the like. Mixtures of cosolvents may be used.

The aqueous medium containing the perhaloalkylsulfonate potassium salt may then be reacted with a tetrasubstituted phosphonium halide. The order of addition does not appear to be important, i.e., reaction may also be accomplished by, for example, dissolving the tetrasubstituted phosphonium halide in an aqueous medium and then adding the perhaloalkylsulfonate potassium salt; by simultaneously dissolving and mixing the reactants; by separately dissolving then mixing the reactants, or the like. The phosphonium sulfonate salts obtained herein may be obtained by using mixtures of perhaloalkylsulfonate potassium salts and tetrasubstituted phosphonium halides.

The processes may be conducted at a broad range of temperatures and reaction times, and will depend on the particular reactants used, stoichiometries of reactants, cosolvent (if present), desired yields, desired purity, cost, convenience, ease of manufacture, and like considerations. For example, temperatures for the various processes may generally be about 10° C. to about 100° C., specifically about 20° C. to about 95° C. more specifically about 30° C. to about 90° C. In one embodiment, the reaction is conducted at elevated temperature, which may generally be 50° C. to about 100° C., more specifically about 75° C. to about 95° C. In another embodiment, the reaction is conducted at room temperature or ambient temperature, which may generally be about 10° C. up to but not including 50° C., more specifically about 15° C. to about 30° C. Likewise, reaction times may vary, but generally may be about 5 minutes to about one day, specifically about 30 minutes to about 12 hours, or more specifically about 60 minutes to about 4 hours. These temperatures and times may be varied greatly and may be determined by those of ordinary skill in the art.

The tetrasubstituted phosphonium halide may used in an at least equimolar amount relative to the perhaloalkylsulfonate salt, and more specifically, the molar ratio of the perhaloalkylsulfonate salt of formula (2) to the tetrasubstituted phosphonium halide of formula (3) may be about 1:1.001 to about 1:1.5, specifically about 1:1.002 to about 1:1.1, more specifically about 1:1.005 to about 1:1.015. The optimum ratio may vary depending on the particular reactants, temperature, cosolvent(s) (if present), and time, and is readily determined by one of ordinary skill in the art.

In another embodiment, the molar ratio of the perhaloalkylsulfonate salt of formula (2) to the tetrasubstituted phosphonium halide of formula (3) may be about 1.001:1 to about 1.5:1, specifically about 1.002:1 to about 1.1:1, more specifically about 1.005:1 to about 1.015:1. The optimum ratio may vary depending on the particular reactants, temperature, cosolvent(s) (if present), and time, and is readily determined by one of ordinary skill in the art.

In a highly advantageous feature, the reactants and aqueous medium are selected so that phosphonium sulfonate salt (1) precipitates from the aqueous medium at high purity, and may be isolated from impurities, in particular halogen-containing impurities and reactants, by simple filtration and washing. It is desirable to remove halogen-containing impurities in particular (such as the tetrasubstituted phosphonium bromide and/or chloride) since these impurities are known to degrade resins such as polycarbonate. Removal of the impurities is readily and efficiently accomplished by washing with water, since the impurities are soluble in water, while the desired product is not.

Other efficient means of removing the impurities comprises dissolving the phosphonium sulfonate salt (1) in aqueous medium at elevated temperatures, specifically about 70° C. to about 100° C., cooling the aqueous medium, collecting the purified phosphonium sulfonate (1) that precipitates or crystallizes from the aqueous medium, and removing residual aqueous medium. A cosolvent may be desired for use in this means of purification, specifically one which is miscible with the aqueous medium and has an effect on the solubility of the phosphonium sulfonate salt (1).

In another embodiment there is provided a method for making the phosphonium sulfonate salts of formula (1) comprising combining, in an aqueous medium, a sulfonylfluoride of formula (4), a tetrasubstituted phosphonium halide of formula (3), and an alkali metal or alkaline earth metal base; and separating the phosphonium sulfonate of formula (1) from the aqueous medium. Specifically, an aqueous medium suitable in this instance is deionized water, substantially free of solvent. Potassium hydroxide is the preferred base. In one embodiment, the reactants and aqueous medium, stoichiometries of reactants, and reaction temperature are selected so that phosphonium sulfonate salt precipitates from the aqueous medium.

Again, the order of addition does not appear to be important. Thus, the components may be mixed simultaneously, or tetrasubstituted phosphonium-halide (3) may be added to an aqueous solution/dispersion of the base, and this medium/dispersion added to a solution/dispersion of sulfonyl fluoride (4). In still another embodiment, sulfonylfluoride (4) and the base are combined, and allowed to react for a time effective to form the alkali sulfonate salt (2). Phosphonium halide (3) is then added to the medium to form the product without isolation of potassium sulfonate salt (2). This method is simple, efficient, and minimizes time and materials. Alternatively, potassium sulfonate salt (2) may be isolated and redissolved with or without cosolvent prior to addition of phosphonium halide (3).

A broad range of reaction times, temperatures, and other process conditions may be used, but about 25° C. (room temperature) to about 100° C. is preferred for ease of manufacture. Optimal reactant ratios are readily determined by one of ordinary skill in the art, and may be, for example, those described above.

Phosphonium sulfonate salt that may be made by the processes described herein include those having the general formula (6): 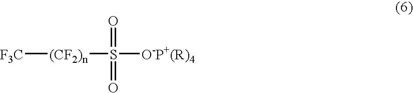
wherein F is fluorine; n is an integer of 0 to about 7, S is sulfur; and each R is the same or different aliphatic hydrocarbon radical containing 1 to about 18 carbon atoms or an aromatic hydrocarbon radical containing 6 to about 18 carbon atoms. In one embodiment, three of the R groups in the organic phosphonium cation may be the same aliphatic hydrocarbon radical containing 1 to about 8 carbon atoms or aromatic hydrocarbon radical containing 6 to about 12 carbon atoms, while the fourth R group may be a hydrocarbon radical containing 1 to about 18 carbon atoms. Anti-static compositions comprising fluorinated phosphonium sulfonates of formula (6) as the principle component thereof may be used in many different ways to make use of their anti-static, compatibility and heat resistance characteristics, for example, in providing such anti-static characteristics to thermoplastic resins. Suitable thermoplastic resins include but are not limited to polycarbonate, polyetherimide, polyester, polyphenylene ether/polystyrene blends, polyamides, polyketones, acrylonitrile-butadiene-styrenes (ABS), or combinations comprising at least one of the foregoing polymers. The phosphonium sulfonate salts are low melting semi-solid materials, and as such, they may be handled as a molten liquid. Some embodiments of the present disclosure are solid crystalline materials at room temperature (about 15 to about 25° C.) and are easy to weigh, handle, and add to the above-described thermoplastic resins.

In addition to the thermoplastic resin, the thermoplastic composition may include various additives ordinarily incorporated in resin compositions of this type. Mixtures of additives may be used. Such additives may be mixed at a suitable time during the mixing of the components for forming the composition. Examples of suitable additives are impact modifiers, fillers, heat stabilizers, antioxidants, light stabilizers, plasticizers, mold release agents, UV absorbers, lubricants, pigments, dyes, colorants, blowing agents, antidrip agents, and flame-retardants.

A common way to practice this method is to add the agent directly to the thermoplastic resin and to mix it at the time of polymer production or fabrication. It may be processed by traditional means, including extrusion, injection, molding, compression molding or casting. The thermoplastic compositions may be manufactured by methods generally available in the art, for example, in one embodiment, in one manner of proceeding, powdered thermoplastic resin, antistatic agent, and/or other optional components are first blended, optionally with chopped glass strands or other fillers in a Henschel high speed mixer. Other low shear processes including but not limited to hand mixing may also accomplish this blending. The blend is then fed into the throat of a twin-screw extruder via a hopper. Alternatively, one or more of the components may be incorporated into the composition by feeding directly into the extruder at the throat and/or downstream through a sidestuffer. Such additives may also be compounded into a masterbatch with a desired polymeric resin and fed into the extruder. The extruder is generally operated at a temperature higher than that necessary to cause the composition to flow. The extrudate is immediately quenched in a water bath and pelletized. The pellets, so prepared, when cutting the extrudate may be one-fourth inch long or less as desired. Such pellets may be used for subsequent molding, shaping, or forming.

The quantity of the phosphonium sulfonate salt added to thermoplastic resin is an amount effective to reduce or eliminate a static charge and may be varied over a range. It has been found that if too little of the anti-static substituted phosphonium sulfonate salt is added to the resin, there still may be a tendency for static charge to build up on an article made of the resin. If the loadings of the anti-static additive become too high, the addition of these quantities is uneconomical, and at some level it may begin adversely to affect other properties of the resin. Thermoplastic compositions with enhanced antistatic properties may be obtained using about 0.01 to about 10 weight percent (wt %), specifically about 0.2 to about 2.0 wt %, more specifically about 0.5 to about 1.5 wt of the anti-static agent with about 90 to about 99.99 wt %, specifically about 99 to about 99.8 wt %, more specifically about 98.5 to about 99.5 wt % polymer, based on the total weight of anti-static agent aid polymer. In one embodiment, in order to obtain a favorable result by such an internal application method in transparent polycarbonate grades, the antistatic agent is used generally in amounts of about 0.01 to about 3.0, specifically about 0.1 to about 1.5 wt. % with respect to the molding composition or specifically in amounts of about 0.4 to about 0.8 wt. %. The antistatic agents provided herein are more strongly resistant against heat and may be added in lower quantities than the traditional ionic surfactants, e.g. phosphonium alkyl sulfonates, and the resin compositions have good transparency and mechanical properties.

The above-described phosphonium salts may further be used to prepare thermoplastic polymer compositions having improved heat stability. In one embodiment a polycarbonate composition comprising an antistatic agent manufactured by one of the above processes has a Yellowness Index of less than about 15, specifically less than about 10, more specifically less than about 8, and even more specifically less than about 6 after aging at 130° C. for 936 hours.

The thermoplastic composition comprising the antistatic agent may be used to form articles such as, for example, computer and business machine housings such as housings for monitors, handheld electronic device housings such as housings for cell phones, electrical connectors, and components of lighting fixtures, ornaments, home appliances, roofs, greenhouses, sun rooms, swimming pool enclosures, carrier tapes for semiconductor package material, automobile parts, and the like.

The thermoplastic compositions may be converted to articles using processes such as film and sheet extrusion, injection molding, gas-assist injection molding, extrusion molding, compression molding, and blow molding. Film and sheet extrusion processes may include and are not limited to melt casting, blown film extrusion and calendaring. Co-extrusion and lamination processes may be used to form composite multi-layer films or sheets. Single or multiple layers of coatings may further be applied to the single or multi-layer substrates to impart additional properties such as scratch resistance, ultra violet light resistance, aesthetic appeal, and the like. Coatings may be applied through application techniques such as rolling, spraying, dipping, brushing, or flow coating. Films or sheets may alternatively be prepared by casting a solution or suspension of the thermoplastic composition in a suitable solvent onto a substrate, belt, or roll followed by removal of the solvent.

Oriented films may be prepared through blown film extrusion or by stretching cast or calendared films in the vicinity of the thermal deformation temperature using conventional stretching techniques. For instance, a radial stretching pantograph may be employed for multi-axial simultaneous stretching; an x-y direction stretching pantograph can be used to simultaneously or sequentially stretch in the planar x-y directions. Equipment with sequential uniaxial stretching sections can also be used to achieve uniaxial and biaxial stretching, such as a machine equipped with a section of differential speed rolls for stretching in the machine direction and a tenter frame section for stretching in the transverse direction.

The thermoplastic compositions of the invention may also be converted to a multiwall sheet comprising a first sheet having a first side and a second side, wherein the first sheet comprises a thermoplastic polymer, and wherein the first side of the first sheet is disposed upon a first side of a plurality of ribs; and a second sheet having a first side and a second side, wherein the second sheet comprises a thermoplastic polymer, wherein the first side of the second sheet is disposed upon a second side of the plurality of ribs and wherein the first side of the plurality of ribs is opposed to the second side of the plurality of ribs.

The films and sheets described above may further be thermoplastically processed into shaped articles via forming and molding processes including, for example thermoforming, vacuum forming, pressure forming, injection molding, and compression molding. Multi-layered shaped articles may also be formed by injection molding a thermoplastic resin onto a single or multi-layer film or sheet substrate, for example by providing a single or multi-layer thermoplastic substrate having optionally one or more colors on the surface, for instance, using screen printing or a transfer dye; conforming the substrate to a mold configuration such as by forming and trimming a substrate into a three dimensional shape and fitting the substrate into a mold having a surface which matches the three dimensional shape of the substrate; injecting a thermoplastic resin into the mold cavity behind the substrate to (i) produce a one-piece permanently bonded three-dimensional product or (ii) transfer a pattern or aesthetic effect from a printed substrate to the injected resin and remove the printed substrate, thus imparting the aesthetic effect to the molded resin.

Those skilled in the art will also appreciate that known curing and surface modification processes, including but not limited to heat-setting, texturing, embossing, corona treatment, flame treatment, plasma treatment, and/or vacuum deposition may further be applied to the above articles to alter surface appearances and impart additional functionalities to the articles.

Accordingly, another embodiment of the invention relates to articles, sheets, and films prepared from the above thermoplastic compositions.

The above processes may be used to form phosphonium salts (1) in an expedited manner and in high purity. In one embodiment, the total amount of ionic impurities is less than about 650 parts per million (ppm), more specifically less than about 500 ppm, even more specifically less than about 100 ppm, more specifically less than about 50 ppm, and most specifically less than about 10 ppm. In another embodiment, the products contain less than about 5 ppm of alkali metals, preferably less than about 4 ppm of alkali metals. In another embodiment, the products contain less than about 500 ppm; preferably less than about 100 ppm, more preferably less than about 50 ppm, and most preferably less than about 10 ppm of halide. Other ionic contaminants, for example phosphate or sulfate, are individually present in amounts of less than about 100 ppm, preferably less than about 50 ppm, most preferably less than about 10 ppm.

The methods are further illustrated by the following non-limiting examples.

### EXAMPLES

Melting points of examples were determined using differential scanning calorimetry (DSC) measurements, conducted by scanning the sample from 50° C. to 100° C. with a scan speed of 10° C./min. Ion content of the salts was determined by ion chromatography (IC).

In the following examples, “MQ water” refers to water deionized and processed through a MilliQ® System. (MilliQ® is a trademark of Millipore Corporation.). The tetraalkylphosphonium haloalkylsulfonate compound demonstrated in the examples was prepared using different starting materials according to the methods described in examples 1-10, below. Table 1, below, provides a listing of the chemicals used in and resulting from the preparation of the examples. The corresponding abbreviated form of the chemical names is given where appropriate.

TABLE 1

| Chemical name | Abbreviation |
|---|---|
| Perfluorobutane sulfonyl fluoride | PFSF |
| Potassium hydroxide | KOH |
| Tetrabutylphosphonium, bromine salt | TBPBr |
| Tetrabutylphosphonium, hydroxide salt | TBPOH |
| MilliQ ® 15-18 Ω deionized water | MQ water |
| Ethanol | EtOH |
| Dichloromethane | CH₂Cl₂ |
| Perfluorobutane sulfonate, potassium salt | K Rimar |
| Tetrabutylphosphonium perfluorobutane sulfonate | TBPPBS |

The solubility of the potassium salt of perfluorobutanesulfonic acid, K Rimar, is described in Table 2.

TABLE 2

| | Concentration of K Rimar in water. | | | | | | |
|---|---|---|---|---|---|---|---|
| | CF₃CF₂CF₂CF₂SO₃^{- +}K (g/10 ml). | | | | | | |
| | 0.1 | 0.2 | 0.5 | 1.0 | 2.0 | 3.0 | 5.0 |
| 20° C. (RT) | s | s | s | i | i | i | i |
| 50° C. | s | s | s | s | i | i | i |
| 80° C. | s | s | s | s | s | s | s |

(s = soluble; i = insoluble.)
K Rimar is soluble at higher concentrations at elevated temperatures, and in relatively low concentrations (less than about 0.5 g at 20° C. (RT).

### Comparative Example 1

Preparation of tetrabutylphosphonium perfluorobutane sulfonate (TBPPBS) using perfluorobutane sulfonyl fluoride and tetrabutylphosphoniumbromide in EtOH/H₂O at 85° C. A portion of 5.00 gram (16.55 mmol) of PFSF was placed in a 100 ml 2-neck round bottom flask, stirred at 85° C. A 50 wt % KOH solution in water (4.46 grams, 39.72 mmol of KOH) was added slowly. During the addition a white solid formed. The resulting reaction mixture was stirred for another hour at 85° C. To obtain a clear solution 75 ml of an EtOH/MQ water mixture (volume ratio EtOH:MQ water=3:4) was added. Next 5.56 gram (16.38 mmol) of TBPBr was dissolved in 25 ml MQ water. The TBPBr solution was poured gradually to the reaction mixture, with stirring. Stirring was continued for an additional 15 minutes at 85° C., post addition. The reaction mixture was then cooled to room temperature (20° C.) and the target product was extracted with 75 ml dichloromethane. The dichloromethane extracts were washed 3 times with 50 ml MQ water. The organic layer solvent was removed by rotary evaporation (50° C., 125 mbar), and the resulting white solid was dried overnight at 50° C. under reduced pressure.

Further purification was done by dispersing the isolated white powder in 100 ml MQ water and heat the dispersion up to 80° C. with stirring. Stirring was continued for 5 minutes and a hazy solution was observed. The dispersion was then cooled to room temperature (20° C.) and a solid white material crystallized. This white material was isolated and dried overnight at 50° C. under reduced pressure. Yield: 65.4%; Mp: 73.6° C.

### Example 2

Preparation of TBPPFS using perfluorobutane sulfonyl fluoride and tetrabutylphosphoniumbromide in H₂O at 85° C. A portion of PFSF (5.00 gram, 16.55 mmol) was placed in a 100 ml 2-neck round bottom flask, and stirred at 85° C. A 50 wt % KOH solution in water (4.46 g, 39.72 mmol of KOH) was added slowly. During the addition a white solid formed. The resulting reaction mixture was stirred for another hour at 85° C. To obtain a clear solution, 50 ml MQ water was added. Next, 5.56 gram (16.38 mmol) of TBPBr was dissolved in 25 ml MQ water. The TBPBr solution was poured gradually into the reaction mixture, with stirring. Stirring was continued for an additional 15 minutes at 85° C., post addition. The reaction mixture was then cooled to room temperature (20° C.), and the precipitated white solid was collected and dried overnight at 50° C. under reduced pressure.

Further purification was done by dispersing the isolated white powder in 100 ml MQ water and heat the dispersion up to 80° C. with stirring. Stirring was continued for 5 minutes and a hazy solution was observed. The dispersion was then cooled to room temperature (20° C.) and a solid white material crystallized. This white material was isolated and dried overnight at 50° C. under reduced pressure. Yield: 44.9%; Mp: 74.3° C.

### Comparative Example 3

Preparation of TBPPFS using perfluorobutane sulfonyl fluoride and tetrabutylphosphoniumbromide in ETOH/H₂O at RT (20° C.). A portion of K Rimar (6.06 gram, 17.9 mmol) was dissolved at room temperature (20° C.) in 75 ml of an EtOH/MQ water mixture (volume ratio EtOH:MQ water=3:4). Separately, TBPBr (6.01 g, 17.7 mmol) was dissolved in 25 ml of MQ water, and was subsequently poured gradually into the solution of K Rimar, with stirring. After addition, the reaction mixture was stirred for an additional 15 minutes. The target product was extracted with 75 ml of dichloromethane, which was in turn washed three times with 50 ml of MQ water. The organic layer solvent was removed by rotary evaporation (50° C. 125 mbar), and the resulting white solid was dried overnight at 50° C. under reduced pressure.

Further purification was done by dispersing the isolated white powder in 100 ml MQ water and heat the dispersion up to 80° C. with stirring. Stirring was continued for 5 minutes and a hazy solution was observed. The dispersion was then cooled to room temperature (20° C.) and a solid white material crystallized. This white material was isolated and dried overnight at 50° C. under reduced pressure. Yield: 89.1%; Mp: 75.6° C.

### Example 4

Preparation of TBPPFS using perfluorobutane sulfonate, potassium salt (K Rimar) and tetrabutylphosphoniumbromide in H₂O at 85° C. A portion of K Rimar (6.06 gram, 17.9 mmol) was dissolved in 30 ml of MQ water at 85° C. Separately, TBPBr (6.01 g, 17.7 mmol) was dissolved in 25 ml of MQ water, and was subsequently poured gradually into the solution of K Rimar at 85° C., with stirring. After addition, the reaction mixture was stirred for an additional 15 minutes. The reaction mixture was then cooled to room temperature (20° C.), and the precipitated white solid was collected and dried overnight at 50° C. under reduced pressure.

Further purification was done by dispersing the isolated white powder in 100 ml MQ water and heat the dispersion up to 80° C. with stirring. Stirring was continued for 5 minutes and a hazy solution was observed. The dispersion was then cooled to room temperature (20° C.) and a solid white material crystallized. This white material was isolated and dried overnight at 50° C. under reduced pressure. Yield: 92.0%; Mp: 75.2° C.

### Example 5

Preparation of TBPPFS using perfluorobutane sulfonate, potassium salt (K Rimar) and tetrabutylphosphoniumbromide in H₂O RT (20° C.). A portion of K Rimar (6.06 gram, 17.9 mmol) was dispersed at room temperature (20° C.) in 30 ml of MQ water. Separately, TBPBr (6.01 g, 17.7 mmol) was dissolved in 25 ml of MQ water, and was subsequently poured gradually into the solution of K Rimar salt dispersion, with stirring. After addition, the reaction mixture was stirred for an additional 15 minutes. The resulting white solid was isolated and dried overnight at 50° C. under reduced pressure.

Further purification was done by dispersing the isolated white powder in 100 ml MQ water and heat the dispersion up to 80° C. with stirring. Stirring was continued for 5 minutes, and a hazy solution was observed. The dispersion was then cooled to room temperature (20° C.) and a solid white material crystallized. This white material was isolated and dried overnight at 50° C. under reduced pressure. Yield: 61.3%; Mp: 75.5° C.

### Example 6

Preparation of TBPPFS using perfluorobutane sulfonate, potassium salt (K Rimar) and tetrabutylphosphoniumbromide in H₂O RT (20° C.). A portion of K Rimar (3.03 gram, 8.95 mmol) was dispersed at room temperature (20° C.) in 30 ml of MQ water. Separately, TBPBr (6.01 g, 17.7 mmol) was dissolved in 25 ml of MQ water, and was subsequently poured gradually into the solution of K Rimar salt dispersion, with stirring. After addition, the reaction mixture was stirred for an additional 15 minutes. The resulting white solid was isolated and dried overnight at 50° C. under reduced pressure.

Further purification was done by dispersing the isolated white powder in 100 ml MQ water and heat the dispersion up to 80° C. with stirring. Stirring was continued for 5 minutes and a hazy solution was observed. The dispersion was then cooled to room temperature (20° C.) and a solid white material crystallized. This white material was isolated and dried overnight at 50° C. under reduced pressure. Yield: 57.6%; Mp: 75.7° C.

### Example 7

Preparation of TBPPFS using perfluorobutane sulfonate, potassium salt (K Rimar) and tetrabutylphosphoniumbromide in H₂O RT (20° C.) ([K Rimar] to [TBPBr]=1:0.9). A portion of K Rimar (6.06 gram, 17.9 mmol) was dispersed at room temperature (20° C.) in 30 ml of MQ water. Separately, TBPBr (5.47 g, 16.1 mmol was dissolved in 25 ml of MQ water, and was subsequently poured gradually into the solution of K Rimar salt dispersion, with stirring. After addition, the reaction mixture was stirred for an additional 15 minutes. The resulting white solid was isolated and dried overnight at 50° C. under reduced pressure.

Further purification was done by dispersing the isolated white powder in 100 ml MQ water and heat the dispersion up to 80° C. with stirring. Stirring was continued for 5 minutes and a hazy solution was observed. The dispersion was then cooled to room temperature (20° C.) and a solid white material crystallized. This white material was isolated and dried overnight at 50° C. under reduced pressure. Yield: 86.7% Mp: 75.5° C.

### Example 8

Preparation of TBPPFS using perfluorobutane sulfonate, potassium salt (K Rimar) and tetrabutylphosphoniumbromide in H₂O RT (20° C.) ([K Rimar] to [TBPBr]=1:1). A portion of K Rimar (6.06 gram, 17.9 mmol) was dispersed at room temperature (20° C.) in 30 ml of MQ water. Separately, TBPBr (6.08 g, 17.9 mmol) was dissolved in 25 ml of MQ water, and was subsequently poured gradually into the solution of K Rimar salt dispersion, with stirring. After addition, the reaction mixture was stirred for an additional 15 minutes. The resulting white solid was isolated and dried overnight at 50° C. under reduced pressure.

Further purification was done by dispersing the isolated white powder in 100 ml MQ water and heat the dispersion up to 80° C. with stirring. Stirring was continued for 5 minutes and a hazy solution was observed. The dispersion was then cooled to room temperature (20° C.) and a solid white material crystallized. This white material was isolated and dried overnight at 50° C. under reduced pressure. Yield: 70.5%; Mp: 75.6° C.

### Example 9

Preparation of TBPPFS using perfluorobutane sulfonate, potassium salt (K Rimar) and tetrabutylphosphoniumbromide in H₂O RT (20° C.) ([K Rimar] to [TBPBr]=1.0:1.1). A portion of K Rimar (606 gram, 17.9 mmol) was dispersed at room temperature (20° C.) in 30 ml of MQ water. Separately, TBPBr (6.69 g, 19.7 mmol) was dissolved in 25 ml of MQ water, and was subsequently poured gradually into the solution of K Rimar salt dispersion, with stirring. After addition, the reaction mixture was stirred for an additional 15 minutes. The resulting white solid was isolated and dried overnight at 50° C. under reduced pressure.

Further purification was done by dispersing the isolated white powder in 100 ml MQ water and heat the dispersion up to 80° C. with stirring. Stirring was continued for 5 minutes and a hazy solution was observed. The dispersion was then cooled to room temperature (20° C.) and a solid white material crystallized. This white material was isolated and dried overnight at 50° C. under reduced pressure. Yield: 65.9%; Mp: 75.7° C.

A commercial sample of perfluorobutanesulfonate tetrabutyl phosphonium salt (from Dupont under the trade name Zonyl® FASP-1) was analyzed for comparison purposes.

The general differences in the preparation of examples 1-10 regarding choice of solvent, reaction temperature, and the ratio of K Rimar to TBPBr (where used) is summarized in Table 1, below. In addition, a summary of the melting points of the isolated products and the yields is also given.

TABLE 3
Yield and melting points of Examples 1-10.

| | | Example No. | | | | | | | | | |
|---|---|---|---|---|---|---|---|---|---|---|---|
| | Units | 1* | 2 | 3* | 4 | 5 | 6 | 7 | 8 | 9 | 10 |
| Solvent | Type | EtOH/H₂O | H₂O | EtOH/H₂O | H₂O | H₂O | H₂O | H₂O | H₂O | H₂O | n.a. |
| Reaction Temperature | ° C. | 85 | 85 | 20 | 85 | 20 | 20 | 20 | 20 | 20 | n.a. |
| Ratio of K Rimar to TBPBr | — | n.a. | n.a. | 1.01:1 | 1.01:1 | 1.01:1 | 0.51:1 | 1.11:1 | 1:1 | 0.91:1 | n.a. |
| Yield | % | 65.4 | 44.9 | 89.1 | 92.0 | 61.3 | 57.6 | 86.7 | 70.5 | 65.9 | n.a. |
| mp | ° C. | 73.6 | 74.3 | 75.6 | 75.2 | 75.5 | 75.7 | 75.5 | 75.6 | 75.7 | n.a. |

*Comparative Example

Purity of examples 1-10, as measured by the amount of residual ions (parts per million or ppm), is shown in Table 4.

TABLE 4

| | Example No. (values shown are in ppm) | | | | | | | | | |
|---|---|---|---|---|---|---|---|---|---|---|
| Ion | 1* | 2 | 3* | 4 | 5 | 6 | 7 | 8 | 9 | 10 |
| Li⁺ | <1 | <1 | <1 | <1 | <1 | <1 | <1 | <1 | <1 | <1 |
| Na⁺ | <2 | <2 | <2 | <2 | <2 | <2 | <2 | <2 | <2 | <2 |
| K⁺ | <2 | <2 | <2 | <2 | 11 | 10 | 11 | 16 | 16 | 2.1 |
| F⁻ | <1 | 14 | <1 | <1 | <1 | <1 | <1 | <1 | <1 | <2 |
| Cl⁻ | <2 | <2 | <2 | <2 | <2 | <2 | <2 | <2 | <2 | <2 |
| Br⁻ | 81 | 16 | <1 | <2 | 4.9 | 3.8 | <2 | 4.8 | 5.2 | <4 |

*Comparative example

It is possible to synthesize the antistatic agent according to all the examples as described above. Impurities can be washed out easily by washing the antistatic agent in water at 80° C. At that temperature the antistatic agent is molten and has a bigger surface area that makes contact with the water then when it is put in there as a solid. The synthesis according to Example 4 is particularly advantageous, in that this example gives both a high yield and high purity (as evidenced by the melting point), while additionally comprising simple synthetic steps.

The singular forms “a,” “an,” and “the” include plural referents unless the context clearly dictates otherwise. The endpoints of all ranges reciting the same characteristic are combinable and inclusive of the recited endpoint. All references are incorporated herein by reference.

While typical embodiments have been set forth for the purpose of illustration, the foregoing descriptions should not be deemed to be a limitation on the scope herein. Accordingly, various modifications, adaptations, and alternatives may occur to one skilled in the art without departing from the spirit and scope herein.

What is claimed is:

1. A method for making a phosphonium sulfonate salt comprising:
combining, in aqueous medium, an alkali metal or alkaline earth metal base, with a compound of the generic formula (4): 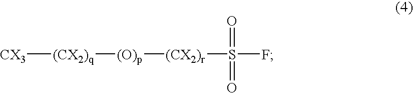
wherein X is independently selected from halogen or hydrogen provided that the molar ratio of halogen to hydrogen is greater than about 0.90; p is 0 or 1, q and r are integers of 0 to about 7, provided that q+r is less than 8 and that if p is not zero then r is greater than zero; and
adding to the product of this addition a stoichiometric excess of a compound of the generic formula (3) to form a precipitate:
(R)₄P-Z (3)
wherein Z is a halogen and each R is the same or different aliphatic hydrocarbon radical containing 1 to about 18 carbon atoms or an aromatic hydrocarbon radical containing about 6 to about 18 carbon atoms, and
separating the precipitated product from the aqueous medium, wherein the precipitated product comprises a phosphonium sulfonate salt of formula (1):

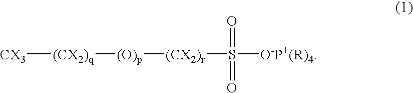

2. The method of claim 1, wherein three of the R in formula (3) are the same radical selected from aliphatic hydrocarbon radicals containing 1 to about 8 carbon atoms and aromatic hydrocarbon radicals containing 6 to about 12 carbon atoms, and the fourth R group is a hydrocarbon radical containing 1 to about 18 carbon atoms.

3. The method of claim 1, where the aqueous medium is substantially free of a cosolvent.

4. The method of claim 1, wherein the alkali metal or alkaline earth metal base is potassium hydroxide.

5. The method of claim 1, wherein the phosphonium sulfonate salt of formula (1) comprises a perfluorinated organic sulfonate anion and an organic phosphonium cation.

6. The method of claim 1, wherein the perfluorinated organic sulfonate anion is selected from the group consisting of perfluoromethane sulfonate, perfluoroethane sulfonate, perfluoropropane sulfonate, perfluorobutane sulfonate, perfluoropentane sulfonate, perfluorohexane sulfonate, perfluoroheptane sulfonate, perfluorooctane sulfonate, and a combination comprising at least one of the foregoing perfluorinated organic sulfonate anions.

7. The method of claim 1, wherein the organic phosphonium cation is selected from the group consisting of tetramethyl phosphonium, tetraethyl phosphonium, tetrabutyl phosphonium, triethylmethyl phosphonium, tributylmethyl phosphonium, tributylethyl phosphonium, trioctylmethyl phosphonium, trimethylbutyl phosphonium trimethyloctyl phosphonium, trimethyllauryl phosphonium, trimethylstearyl phosphonium, triethyloctyl phosphonium and aromatic phosphoniums such as tetraphenyl phosphonium, triphenylmethyl phosphonium, triphenylbenzyl phosphonium, tributylbenzyl phosphonium and a combination comprising at least one of the foregoing perfluorinated organic phosphonium cations.

8. The method of claim 1, wherein X is Fl.

9. The method of claim 1, wherein Z is Br or Cl.

10. The method of claim 1, wherein the product of the addition of an alkali metal or alkaline earth metal base with an aqueous solution of a compound of the generic formula (4) is a corresponding potassium sulfonate salt of the generic formula: 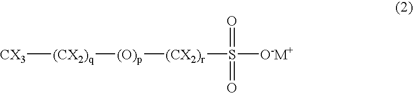
wherein M is an alkali metal or alkaline earth metal.

11. The method of claim 10, wherein M is K.

12. The method of claim 1, wherein X is Fl and Z is Br or Cl.

13. The method of claim 12, wherein three of the R in formula (1) are the same or different aliphatic hydrocarbon radical containing 1 to about 8 carbon atoms or aromatic hydrocarbon radical containing 6 to about 12 carbon atoms, and the fourth R group is a hydrocarbon radical containing 1 to about 18 carbon atoms.

14. The method of claim 1, wherein the product contains less than 650 parts per million of ionic impurities.

15. The method of claim 14, wherein the ionic impurities are soluble in water.

16. The method of claim 14, wherein the product contains less than 500 parts per million ionic impurities.

17. The method of claim 16, wherein the product contains less than 100 parts per million ionic impurities.

18. The method of claim 1, wherein the product contains less than 5 parts per million of alkali metals.

19. The method of claim 1, wherein the product contains less than 500 parts per million of halide.

20. A method for making a phosphonium sulfonate salt comprising:
combining, in aqueous medium, potassium hydroxide with a compound of the generic formula (4): 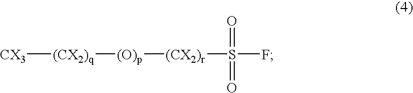
wherein X is independently selected from halogen or hydrogen provided that the molar ratio of halogen to hydrogen is greater than about 0.90; p is 0 or 1, q and r are integers of 0 to about 7, provided that q+r is less than 8 and that if p is not zero then r is greater than zero; and
adding to the product of this addition a stoichiometric excess of a compound of the generic formula (3):
(R)₄P-Z (3)
wherein Z is Br or Cl, and each R is the same or different aliphatic hydrocarbon radical containing 1 to about 18 carbon atoms or an aromatic hydrocarbon radical containing about 6 to about 18 carbon atoms, and
separating the precipitated product from the aqueous medium, wherein the precipitated product comprises a phosphonium sulfonate salt of formula (1): 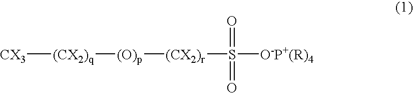
and wherein the precipitated product contains less than 100 parts per million ionic impurities, less than 5 parts per million of alkali metals, and less than 100 parts per million halide.
